(19) United States
(12) Patent Application Publication
(10) Pub. No.: US 2024/0165308 A1
(43) Pub. Date: May 23, 2024

(54) SYSTEM AND METHOD FOR PREVENTING PATIENT REACTIONS DURING BLOOD DRAW

(71) Applicant: Fenwal, Inc., Lake Zurich, IL (US)

(72) Inventors: James Madsen, Chicago, IL (US); Winston Ding, Evanston, IL (US)

(21) Appl. No.: 18/512,012

(22) Filed: Nov. 16, 2023

Related U.S. Application Data
(60) Provisional application No. 63/384,327, filed on Nov. 18, 2022.

(51) Int. Cl. A61M 1/02

(52) U.S. Cl. CPC A61M 1/02; A61M 2205/18; A61M 2205/3313; A61M 2205/50; A61M 2230/50

(57) ABSTRACT

A method and system for drawing and collecting blood and/or blood components is disclosed. The method and system include a patient temperature monitoring device for monitoring a patient's temperature throughout a collection procedure, which includes an infrared camera. The infrared camera may be calibrated regularly to ensure accurate measurements.

### CROSS-REFERENCE TO RELATED APPLICATIONS

This application claims the benefit of and priority of U.S. Provisional Patent Application Ser. No. 63/384,327, filed Nov. 18, 2022, the contents of which are incorporated by reference herein.

### FIELD OF THE INVENTION

The present disclosure is directed to a blood draw system and a method for anticipating and preventing patient or donor reactions. The system monitors patient temperature and can alter or end a procedure before a patient reaction. The system includes an infrared camera which is regularly calibrated to ensure accurate temperature measurements.

### BACKGROUND

Blood may be drawn from the arm or hand of a patient or donor for any of a number of reasons. Whole blood may be collected and donated to hospitals or other patient care centers. Alternatively, whole blood may be collected and separated into its components and the components may also be collected, while returning other components to the patient. The process of withdrawing blood, collecting some blood components and returning other components is commonly referred to as apheresis. During apheresis procedures, for example, blood is drawn from a patient or donor, a specific blood component is separated from the whole blood and removed and/or collected, and the remaining blood components are returned to the patient or donor, all in a continuous flow mode.

Apheresis and other blood draw procedures can range from approximately 30 minutes to 10 hours. During a blood donation procedure such as, but not limited to apheresis, patients can experience reactions caused by physiological factors or sudden intravascular depletion. Specifically, patients can experience vasovagal reactions or vasoconstriction that can cause sweating, nausea, dizziness, and/or fainting. During an apheresis procedure, citrate toxicity can also occur if the amount of citrate (which is typically present in the anticoagulant used in blood collection and processing) returned to the patient is too high for the patient's system to process effectively. Currently, reaction management or prevention strategies are implemented on a patient-specific basis by the phlebotomists, which therefore rely on the patient reporting early symptoms of a reaction and the phlebotomist taking time to act on those. Notwithstanding the best efforts of phlebotomists or blood center operators to address patient reactions, they remain common occurrences and can at times be significant.

Studies have shown that physiologic changes may occur prior to the onset of a full-blown patient reaction, such as changes in temperature of the donor or patient. Specifically, a patient may experience a difference or change in facial temperature. Thus, it would be desirable to provide a system that monitors such temperature changes before a full-blown reaction occurs, and prevents patient reactions.

When providing a system which monitors temperature, such as an infrared camera, it is important that the temperature measurements are accurate. Currently, calibration of infrared cameras is a one-time factory calibration. However, in order to ensure the accuracy of a patient monitoring device, it is desirable for any camera used for a patient monitoring device to be calibrated more regularly so that the measurements are more accurate.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a method for reducing patient reactions during a blood draw using a blood draw system with a blood collection unit and a controller includes establishing a baseline temperature for a patient and initiating a blood draw. The method further includes measuring patient temperature with at least one patient temperature monitoring device during the blood draw for a measured patient temperature and comparing the measured patient temperature to the baseline temperature. The method also includes predicting whether a patient reaction is likely to occur and stopping the blood draw if a patient reaction is likely to occur. The steps of the method are performed until the blood draw is complete.

In another aspect, a system for performing a blood draw procedure includes a vein access device and a fluid circuit comprising tubing connected to a vein access device and at least one blood collection container for collecting blood and/or blood components. The system also includes at least one patient temperature monitoring device and an indicator. The system further includes a controller configured to communicate with the patient temperature monitoring device and an indicator. The controller is configured to establish a baseline temperature for a patient, instruct the patient temperature monitoring device to measure patient temperature during the blood draw and record the measured temperature, compare the measured temperature to the baseline temperature, predict whether a patient reaction is likely to occur, and initiate an alarm at the indicator in order to stop the blood draw if a patient reaction is likely to occur. These steps are repeated until the blood draw is completed.

In a further aspect, a blood draw system includes a plurality of blood collection units, a patient temperature monitoring device, an indicator, and a controller. Each blood collection unit includes a fluid circuit and at least one blood collection container. The controller is associated with each blood collection unit and communicates with the patient temperature monitoring device and an indicator. The controller is configured to establish a baseline temperature for a patient, instruct the patient temperature monitoring device to measure patient temperature during the blood draw and record the measurements, compare the measured temperature to the baseline temperature, predict whether a patient reaction is likely to occur, and initiate an alarm at the indicator in order to stop the blood draw if a patient reaction is likely to occur. These steps are repeated until the blood draw is completed.

In another aspect, a method of calibrating an infrared camera includes capturing an image of an object with a uniform temperature, zooming in on the image to establish a real temperature, zooming out on the image and measuring temperatures across the image, formulating an adjustment equation to correct for temperature outside of the real temperature, based on location from a left side of the infrared camera, and applying the adjustment equation to the camera.

These and other aspects of the present subject matter are set forth in the following detailed description of the accompanying drawings.

### BRIEF DESCRIPTION OF THE DRAWINGS

FIG. 1 is a schematic illustration of a blood draw system of the type that may be advantageously used in accordance with the present disclosure.

FIG. 2 is a schematic illustration of the possible components of a blood draw system of the type that may be advantageously used in accordance with the present disclosure.

FIG. 3 is a schematic illustration of face sectioning performed by at least one camera in accordance with the present disclosure.

FIG. 4 is a schematic illustration of the pixel mapping between two cameras in accordance with the present disclosure.

FIG. 5a is an example of coordinate mapping in accordance with the present disclosure.

FIG. 5b is an example of coordinate mapping in accordance with the present disclosure.

FIG. 6 is an example of thermal tracking of a patient's forehead.

FIG. 7 is an illustration of the calibration process in accordance with the present disclosure.

FIG. 8 is an example of a graph plotting the temperature measurements and the calculated adjustment formula in accordance with the present disclosure.

### DETAILED DESCRIPTION

The embodiments disclosed herein are for the purpose of providing an exemplary description of the present subject matter. They are, however, only exemplary and not exclusive, and the present subject matter may be embodied in various forms. Therefore, specific details disclosed herein are not to be interpreted as limiting the subject matter as defined in the accompanying claims.

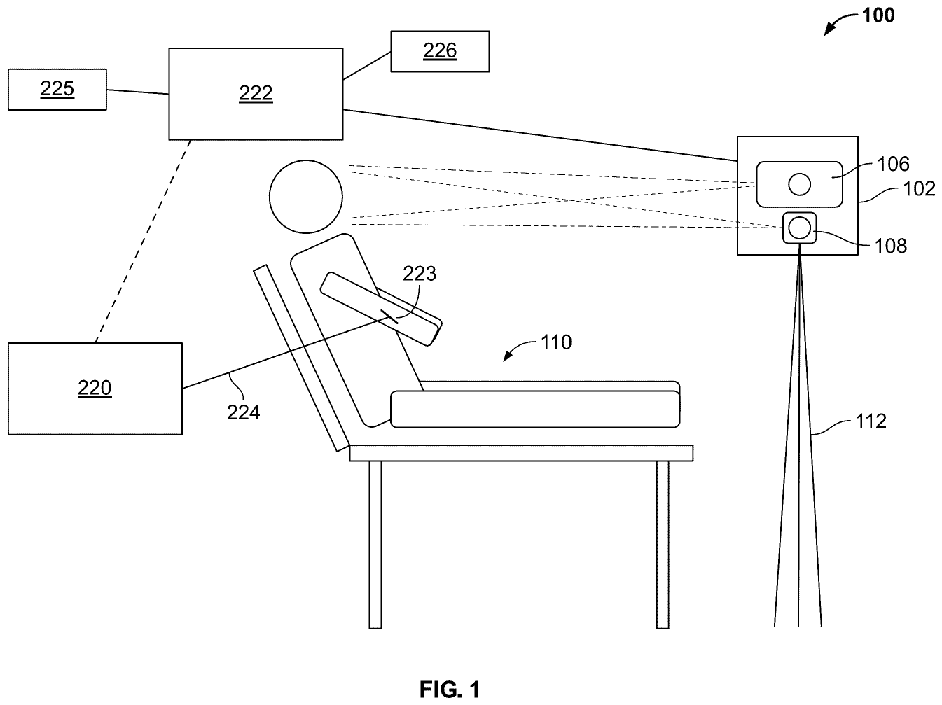

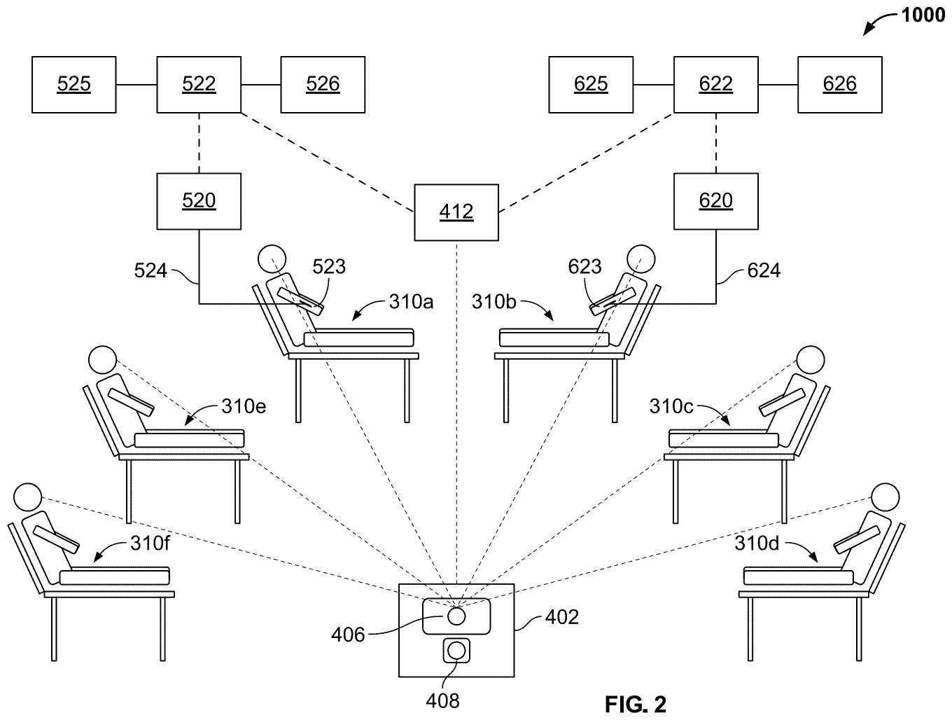

FIGS. 1-2 show components of a blood draw system that embody various aspects of the present subject matter described herein. “Blood” includes without limitation whole blood and/or blood components, such as red cells, white cells, plasma, and platelets. The blood draw may include whole blood collection or collection of one blood component and return of other components.

The blood draw system described herein will typically employ a disposable, single-use fluid processing assembly, module or circuit through which the fluid(s) flow, and reusable components to which the disposable is mounted or otherwise cooperatively engaged to control the flow of fluids through the disposable module and the drawing of the blood or blood components. The blood draw system also includes reusable components which are used to monitor the patient during a blood draw.

Depicted in the figures are two different schematics for different embodiments of the current blood draw system. FIG. 1 depicts a single patient and blood collection unit, and FIG. 2 depicts a plurality of patients and blood collection units (one associated with each patient or donor). The blood collection units can be used for the collection of whole blood donation or the collection of whole blood and separation thereof into blood component donation (with return of remaining components to the patient), such as by apheresis. There may be overlap in components of the system and like numbers (for example, 222 and 522) indicate similar or identical components.

The draw system 100, as shown for illustration only, comprises a whole blood draw or flow path or line 224 for fluid flow from the vascular system of the donor/patient via a vein access device (such as a needle) 223 at the donor/patient 110 to the blood collecting unit 220. The system 100 further includes a controller 222, input 226 and indicator 225, which although illustrated together, may be separate, and a patient temperature monitoring device 102. Patient temperatures, such as of the face, are monitored and the system may alert a phlebotomist or patient that a reaction is likely to occur or alternatively cause the blood collecting unit to stop or pause the blood draw.

The draw system 1000, as shown in FIG. 2 for illustration only, comprises blood collecting units (520 and 620 are shown, but each patient 310 (310a-310f) may be associated with a unit), that are each connected to a patient 310 by a blood draw or flow path or line (524, 624 are shown, but each patient 310 may have a separate line) for fluid flow from the vein access device 523, 623 at the donor/patient 310 to the blood collection unit 520, 620, a patient temperature monitoring device 402, and an optional group computing device 412 that can be optionally connected to each blood collecting unit 520, 620. The patient temperature monitoring device can monitor each patient and provide feedback to an associated controller 522, 622 and alert a phlebotomist/patient through an associated input 526, 626 and/or indicator 525, 625, all as described in more detail below.

The above description of the systems 100 and 1000 are intended to be illustrative and not exhaustive. For example, and as noted above, although two blood collecting units 520, 620 are depicted in FIG. 2, each donor/patient will have a corresponding blood collecting unit and blood flow line 524, 624. Further, the patient temperature monitoring device 402 may be connected to a controller of each blood collecting unit or connected to a group communicating device 412 which communicates with the controller 522, 622 of each blood collecting unit 520, 620. The controller and input may be part of a separate device, or the controller and input may be part of the patient temperature monitoring device. The blood collecting units are generically depicted and other system components, such as a separator, fluid circuit, pumps, valves, source of saline priming fluid, leukocyte reduction filters, sensors and the like, although not illustrated in the figures may be included in the units as desired.

Turning now to the various illustrated components, the vein access device 223, 523, 623 may include, for example, a single vascular access member such as a phlebotomy needle, vascular catheter or other access device for use in single access-site (sometimes called “single-needle”) procedures, in which whole blood is alternately drawn from a patient or donor and blood or blood components are alternately returned. The vein access also may include a pair of such access devices for vascular access (“double needle”) at different locations to permit simultaneous withdrawal of whole blood and return of blood or blood components.

The vein access device 223, 523, 623 is fluidly connected, such as by flexible plastic tubing, with a blood collecting unit 220, 520, 0620 via a whole blood draw or flow line 224, 524, 624 as part of a fluid flow circuit. It may be noted here that the fluid flow lines of the system 100, 1000 may have a variety of configurations and be made of any suitable material. For example, the flow lines may be provided as tubular conduits formed of either flexible tubing or the flow path may be preformed in a rigid plastic flow control cassette that is operated by solenoid, pneumatic or other valve arrangement to control flow direction through the cassette, as illustrated, for example in U.S. Pat. No. 5,538,405 to Patno et al. or U.S. Pat. No. 6,481,980 to Vandlik et al., both of which are incorporated by reference herein, or may be of any other suitable configuration.

The fluid flow circuit or flow set is intended to be a sterile, single use, disposable item. Before beginning a given blood draw procedure, the operator mounts the various components of the fluid flow circuit onto the blood draw system. The controller implements the procedure based upon preset protocols, taking into account other inputs from the operator. Upon completing the procedure, the operator removes the fluid flow circuit from association with the blood draw system. The portions of the fluid flow circuit holding the collected blood component or components (e.g., collection containers or bags) are removed and retained for storage, transfusion, or further processing. The remainder of the fluid flow circuit is removed and discarded.

The blood collecting unit 220, 520, 620 may be variously provided without departing from the scope of the present disclosure, and various devices may, for example, be used to obtain the treatment of blood and/or the separation of blood into its constituents, if desired. The blood collecting unit may simply be a bag or other type of holding container for storing donated blood. However, the blood collecting unit may also be connected to a separator or other blood processing components for separating different blood components. For example, the blood can be processed through a known centrifugal separation chamber, such as employed in the ALYX® or AMICUS® separators marketed by Fenwal, Inc. of Lake Zurich, Illinois, a subsidiary of Fresenius-Kabi of Bad Homburg, Germany, or centrifugal blood separators made by other manufacturers including Haemonetics Inc. of Braintree, Massachusetts; or CaridianBCT of Lakewood, Colorado. Static or moving membrane type separators may also be used to remove a particular blood component or constituent. More specifically, suitable blood collecting units, may include, but are not limited to, the centrifugal or spinning filtration membrane apheresis systems, such as those described in greater detail in U.S. Pat. No. 4,526,515 to De Vries; U.S. Pat. No. 5,194,145 to Schoendorfer; U.S. Pat. No. 6,312,607 to Brown et al.; U.S. Pat. No. 6,524,231 to Westberg et al.; U.S. Pat. No. 4,094,461 to Kellogg et al.; U.S. Pat. No. 7,052,606 to Gibbs et al.; U.S. Pat. No. 4,300,717 to Latham and U.S. Patent Application Publication No. 2009/0215602 to Min et al., all of which are hereby incorporated by reference.

The containers and the plastic tubing of the fluid circuit may be made of conventional medical grade plastic that can be sterilized by sterilization techniques commonly used in the medical field such as, but not limited to, radiation or autoclaving. Plastic materials useful in the manufacture of containers and of the tubing in the circuits disclosed herein include plasticized poly(vinyl chloride). Other useful materials include acrylics. In addition, certain polyolefins may also be used.

A variety of different disposable fluid flow circuits may be used in combination with the blood draw system, with the appropriate fluid flow circuit depending on the donation procedure to be carried out using the system.

The draw system 100, 1000 may include one or more patient temperature monitoring devices to monitor patient temperature. Although different locations on the body patient may be monitored for temperature changes, in an exemplary embodiment, the patient temperature monitoring device 102 monitors the donor or patient's face. In another embodiment, the patient temperature monitoring device 102 monitors the patient's arm, specifically near the donation needle location. The temperature monitoring devices may include at least one camera. In one aspect, the draw system includes at least two different cameras for monitoring temperatures of a patient, at least one digital camera 106 and at least one infrared camera 108. The patient temperature monitoring device 102 may simply include the camera(s) and the structure(s) for mounting the cameras. The patient temperature monitoring device may also include a controller for processing the data from the cameras.

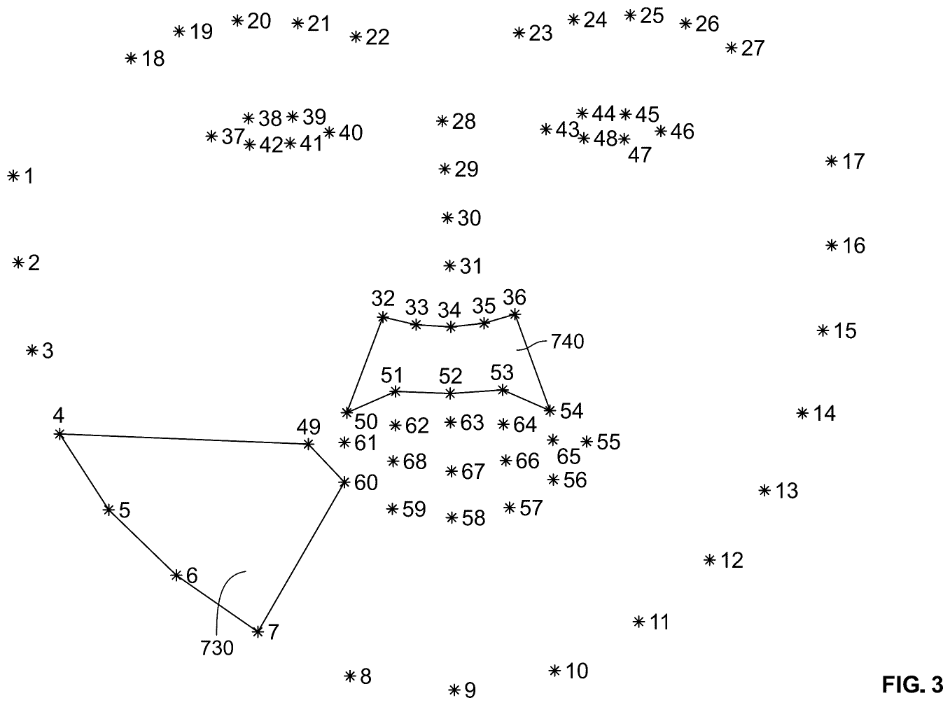

The patient temperature monitoring device 102, 402 may include a standard camera 106, 406, such as any type of point and shoot camera. The camera 106, 406 may be a digital camera or any camera that is capable of producing digital images. The camera 106, 406 may also be part of a phone or a tablet computer that is able to be mounted to a surface and in view of a patient or patients. The standard camera can be used for facial recognition of the patient and for defining specific bounded areas of the face, for example of the forehead. FIG. 3 shows an example of bounding that can be achieved by a camera. Bounded areas 730 and 740 are created for specific portions of the pixels of the face that are mapped (shown as reference numbers 1-68). A specific first, second, or third section of the face may be mapped and repetitively analyzed during a procedure to analyze the temperature of a patient. The section of the face that is mapped and analyzed may include the forehead, a portion of the cheek (such as 730 shown in FIG. 3), part of the face between the nose and mouth (such as 740 shown in FIG. 3), or any other portion of the face. This analysis may include comparing measurements at one section to previous measurements at that section or a baseline temperature for that section. This analysis may also include comparing sections of the face to each other. Additionally, the standard camera may be used to monitor the face and any changes in the facial features of the patient. For instance, the camera may be able to detect changes in facial appearance, such as drooping of one of more portions of the face. In this embodiment, the system may be triggered to create an alarm when the appearance of the face changes.

The patient monitoring device may also include an infrared camera 108. An infrared camera can be used to measure the infrared energy of an object. An infrared camera focuses infrared energy onto a chip that contains thousands of detector pixels arranged in a grid. Each pixel in the array reacts to the infrared energy focused on it and produces an electronic signal. This infrared energy can produce an image that can map temperature across a surface at different pixels. Each temperature value is assigned a different color and typically move toward red as being the warmest and blue as being the coldest. The resulting matrix of colors is sent to memory and to the camera's display as a temperature picture (thermal image) of that object. An infrared camera may be used to monitor temperature across the face or other body parts of a patient.

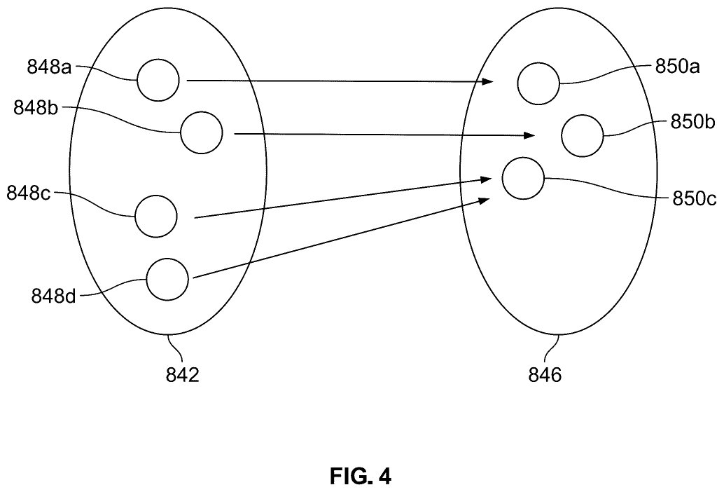

Using both cameras, temperature information for a donor/patient can be tracked. Pixels of interest from the digital or standard camera 106, 406 and the infrared camera 108, 408 can be mapped together. Specifically, as shown in FIG. 4, the digital pixels (848a, 848b, 848c, 848d) from the image 842 from the standard camera can be mapped onto the infrared camera image 846 and its pixels (850a, 850b, 850c, 850d) to determine corresponding thermal pixels. These thermal pixels can then be analyzed to find temperature information. The patient temperature information can be used to track temperature changes over the donation time and/or temperature variations between facial regions. The cameras or an associated controller may record, track, and analyze these values to assess any change for a patient.

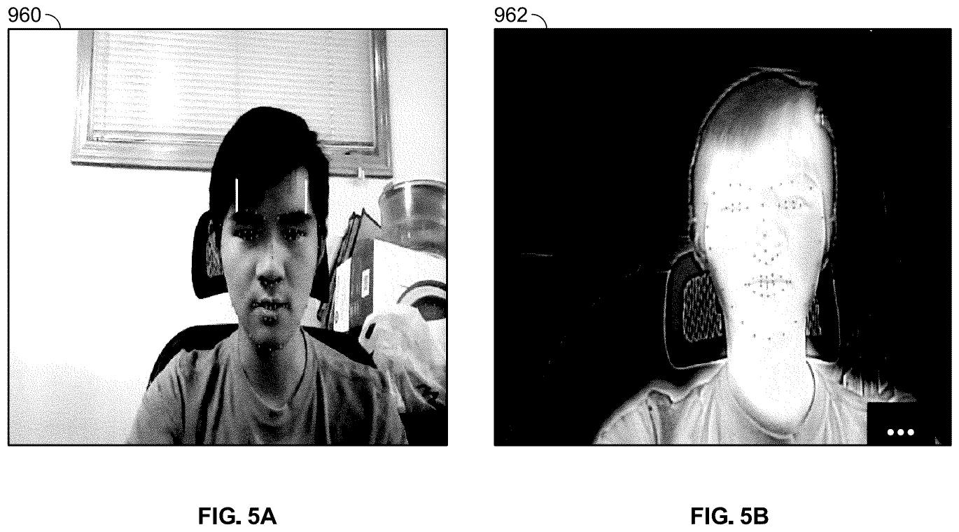

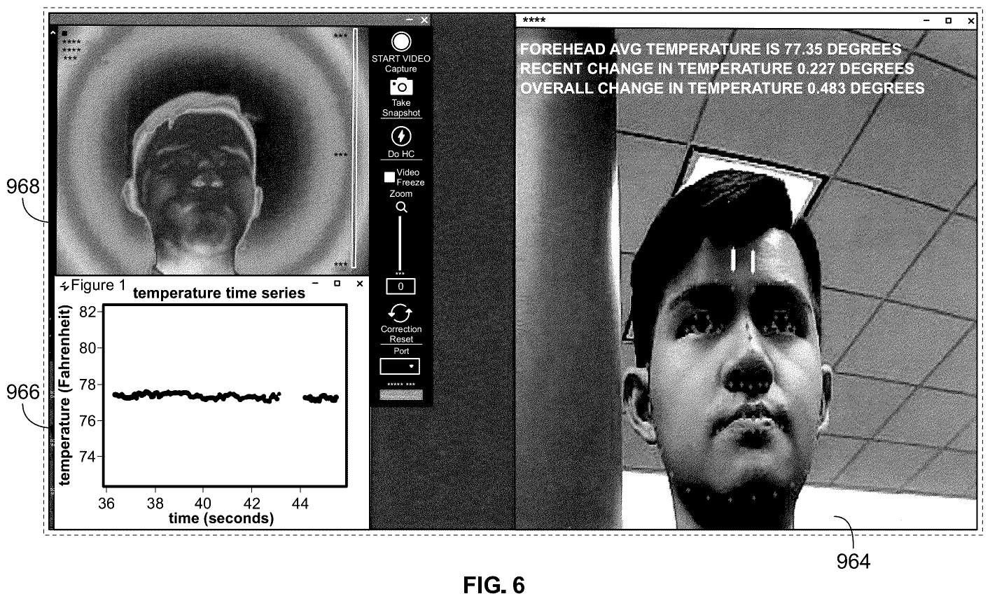

FIG. 5A shows an example of a facial image 960 and pixels produced by a standard camera. FIG. 5B shows an example of a facial image 962 and pixels produced by an infrared camera. FIG. 6 shows an example of the system's produced thermal tracking with the standard camera image 964, infrared camera image 968 and the thermal tracking produced in graph 966.

Although discussed separately, the cameras may be a single camera capable of taking both standard and infrared images. Additionally, multiple standard and multiple infrared cameras may be used in combination.

Because the drawing system and method of monitoring a patient during the blood draw rely heavily on output from an infrared camera 108, 408, it is important that this camera or cameras provide accurate, reliable information. A camera is normally calibrated only once by a manufacturer with a one-time factory calibration. However, due to the reliance on the camera for the system and method, the infrared camera 108, 408 of the embodiments of the current disclosure is calibrated much more frequently. For instance, the camera 108, 408 may be calibrated monthly, daily, hourly, or before each use of the camera (such as with each new patient). The camera can also be calibrated based on number of patients, so the camera may be calibrated after every 10 users, 5 users or even after every user.

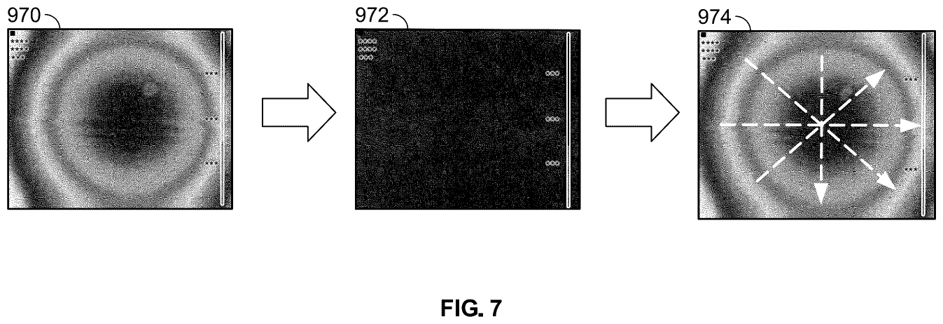

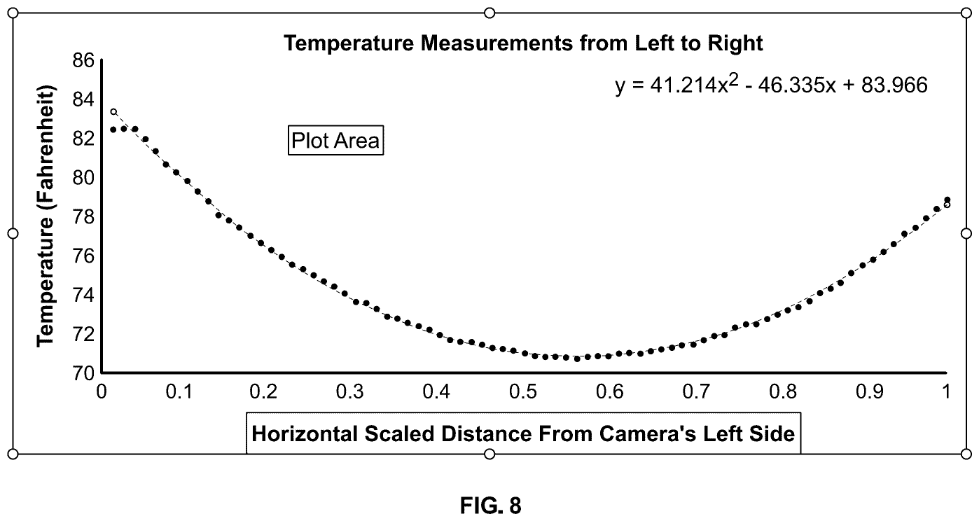

Temperature measurements can vary across the view of the camera when viewing an object of uniform temperature, as shown in FIG. 7. For the first step in calibrating the camera, an object of uniform temperature must be provided for the calibration. Once provided, an image is generated from the infrared camera such as is shown in 970 of FIG. 7. This image shows the inconsistencies in temperature across the image. These measurements will always vary but can be corrected by establishing a relationship between the view on the camera and the correction for distance from one side or point on a camera. In order to correct the inconsistent temperature across the image, an accurate real temperature must be calculated or assessed first. The center view of the camera will display the most precise readings and the lowest measured temperature of the entire view, this lowest temperature is considered the real temperature. The calibration technique can accurately determine the real temperature by zooming in on the same image, such as by 2×, 8× or up to 10×, an example of which is shown in 972 of FIG. 7. The real temperature measurement is then taken from the center of this zoomed in image, creating the basis for the real temperature. The image is returned to normal zoom (1×) and temperature measurements are taken across the image. The temperature measurements are scanned horizontally, vertically and diagonally across the diameter of the image 974, as shown in FIG. 7. The resulting measurements are recorded and inputted to create a mathematical relationship or equation, such as are shown in the graph of FIG. 8. Once this is assessed, the measurements across an image can be corrected based on the distance from the left side of the camera and inserting these values into the calculated equation to get the correct measurement. The calibration is then complete and any measurements taken afterward may be significantly more precise based on this correction.

Cameras may be mounted in any possible fashion. They may be mounted together or separate or simply set up near to each other. Each may have its own tripod or holding structure 112 or each camera may be set up on its own structure or tripod. The cameras may be set up in view of a single patient. A second embodiment in FIG. 2 shows a set of cameras for use by a whole room or section of a donation facility. In the second embodiment the cameras may be placed in at least one location on a ceiling or any other surface which may be in view of the patient(s), with the two cameras placed together or on different parts of the ceiling or other structure. Arrangement of the cameras should be such that they are in view of the patient(s) that is being monitored.

As will be discussed further below, the cameras can be operated by phlebotomist, or may be controlled by a controller. Each camera may have its own controller, or one controller may control all of the cameras of the patient temperature monitoring device. This controller can also process and control the calibrations, record the measurements, and compare and analyze the measurements.

In addition to the cameras of the patient temperature monitoring device, the draw system may include additional devices for monitoring patient parameters. The draw system may include additional monitoring devices to monitor parameters such as blood pressure, heart rate, sweat, facial expression, nerve conduction, and any other parameters for monitoring a person. The type of additional monitoring device(s) utilized in the system will depend on the additional patient parameters being monitored. For instance, for blood pressure monitoring, a cuff or other equivalent device may be used as a sensor. The monitoring device may also be a pulse oximeter, which can detect pulse change and/or oxygen levels in the blood. Additionally, the monitoring device may also be an electrocardiogram (EKG) which can monitor heart activity.

The patient temperature monitoring device may operate automatically and intermittently (such as every 2, 5, or 10 minutes) or continuously as programmed by the controller for monitoring patient parameters.

Upon a notification or alarm, the draw system may trigger a phlebotomist to end a procedure or can utilize an automatic adjustment mechanism to end or adjust the blood draw. The adjustment mechanism may alter the height/position of a component of or of the fluid circuit and/or flow rate through one or more flow lines. The adjustment mechanism can be mechanical, pneumatic, hydraulic or ultrasonic. Any adjustment mechanism can be used that can move or adjust the blood container or flow line. The adjustment mechanism may also be a pump or clamp.

The draw system 10, 100 may also include weight scales for blood or blood component containers and may be provided with a plurality of hooks or supports that may support various components of the fluid flow circuit or other suitably sized and configured objects.

For controlling the draw procedures and processing the information from the patient temperature monitoring device 102, the system 100, 1000 further includes the controller 222, which is configured to control the operation of the system 100, 1000. The controller 222 may be provided as a computer or associated programmable microprocessor or other known mechanism for controlling one or more of the elements and processing information from one or more elements of the system 100, 1000 in accordance with the procedure and steps set forth herein. This controller 222 can be part of the blood collecting unit, part of the patient temperature monitoring device, part of a group computing device or part of a stand-alone component. Alternatively, a plurality of controllers may be employed rather than just a single controller 222. For instance, the patient temperature monitoring device 102 may include a controller with a processor for processing the information from the camera(s) and the blood collecting unit may include a separate controller for communicating with the controller of the patient temperature monitoring device 102, 402.

As is also illustrated throughout the Figures, the controller 222 may be coupled to one or more of the structures described above, for example to receive information (e.g., in the form of signals) from these structures or to provide commands (e.g., in the form of signals) to these structures to control the operation of the structures. As illustrated, the controller 222 may be coupled to at least one input 226, indicator 225, and at least one patient temperature monitoring device 102. Additionally, a controller 222 (when part of the blood collection unit) may be coupled to pumps, and the drive or centrifuge separator (if included in the donation system) to provide commands to those devices to control their operation. The controller 222 may be directly electrically connected to these structures to be coupled to them, or the controller 222 may be directly connected to other intermediate equipment that is directly connected to these structures to be coupled to them. The controller may also be wirelessly connected to any of these devices.

The controller may be connected to at least one input. The at least one input 226, 526, 626 may include a number of different devices according to the embodiments described herein. The input provides a mode of communication from the operator to the device and the device to the operator. For example, the input 226, 526, 626 could include a keyboard, keypad or touchscreen by which a user may provide information and/or instructions to the controller 222. Alternatively, the input 226, 526, 626 may be a touch screen, such as may be used in conjunction with a video display. The input 226, 526, 626 could also include a reader or scanner, such as a barcode reader or scanner or an RFID reader. The assembly of the input/touch screen and video display may be one of the afore-mentioned structures to which the controller 222 is coupled from which the controller 222 receives information and to which the controller 222 provides commands. According to still other embodiments, the input 226 may be in the form of computer equipment that permits the blood draw system including the controller 222 to communicate (whether via wires, cables, etc. or wirelessly) with other systems over a local network, or with other cell processing systems or other computer equipment (e.g., a server) over local networks, wide area networks, or the Internet. According to such an embodiment, the input 226 may include an internal transmitter/receiver device.

Using the input 226, a phlebotomist or operator may enter parameters and information relevant to the blood collection procedure. This information can include patient measurements such as height, weight, hematocrit, or characteristics such as gender, previous donation reactions, or any other relevant information. This information can also include process parameters. A phlebotomist or user may also enter patient specific parameters, including baseline parameters, such as baseline temperature. The baseline temperature may also be measured by the system and recorded. The baseline temperature may have a minimum or maximum and/or acceptable range, which when measured temperatures fall outside this range, the risk of a patient reaction is higher. For example, if the temperature of the patient drops more than 2 degrees Celsius from the baseline temperature value, the patient may experience an adverse event.

The system may also include at least one indicator 225 associated with the controller 222. The indicator 225 may be incorporated anywhere into the system without departing from the scope of the present disclosure, it may be part of the input 226 or a separate component. The indicator 225 is configured to display at least one of an indication of a status of the system, which may include (for example) when the system is ready to begin processing, when the system is processing, when the system has completed processing, and when there has been an error. The indicator 225 may display or represent the status of the draw system in any suitable manner. Additionally, the indicator 225 may include a display or light which indicates a status of the patient parameter being monitored. The display may continually show measured values for the parameter or provide a light or other indication of whether the values measured are within an acceptable range or when the values are outside of an acceptable range. If the property or properties is/are outside of an acceptable range, then the controller 222 may initiate an alarm or error condition to alert an operator to the condition. The alarm may be a notice on a display of the input, a light or color, or a sound alarm.

The system 100, 1000 can also include buttons or icons associated with the controller. The buttons or icons may be variously configured and positioned at any suitable location of the system. One button/icon may be a start button/icon for initializing a blood collection procedure and the other button/icon of the set may be a stop button/icon for stopping a blood collection procedure. These buttons may be integrated within the input or separate for performing separate functions.

The blood collecting device 220, 520, 620 and patient temperature monitoring device 102 communicate with controller 222. The controller 222 carries out process control and monitoring functions for the system 100, 1000. The controller 222 comprises a main processing unit (MPU), which can comprise, e.g., a PENTIUM® type microprocessor made by Intel Corporation, although other types of conventional microprocessors can be used.

According to other embodiments, the controller 222 may include one or more electrical circuits designed to carry out the actions described herein. In addition, the controller 222 may include one or more memories. The instructions by which the microprocessor is programmed may be stored on the memory associated with the microprocessor, which memory/memories may include one or more tangible non-transitory computer readable memories, having computer executable instructions stored thereon, which when executed by the microprocessor, may cause the microprocessors to carry out one or more actions as described below.

The controller 222 is configured and/or programmed to monitor, collect, analyze and relay information about the patient and may also be programmed to operate the blood collecting unit(s) and camera(s). More particularly, in carrying out any one of these blood collecting or blood processing applications, the controller 222 is configured and/or programmed to collect information from the patient temperature monitoring device 102 and blood collecting unit 220 and provide information to a phlebotomist or the blood collecting unit while a blood draw is being done. This may include generating an alarm to notify or a signal to the blood collecting unit to stop the collection. This may also include prompting the infrared camera's calibration before beginning the process of collecting blood and/or when to begin/end capturing images. Hence, while it may be described herein that a particular component of the blood draw system performs a particular function, it should be understood that that component is being controlled by the controller to perform that function.

Before, during, and after a procedure, the controller may receive readings from the patient temperature monitoring device 102 and process this information before communicating with a phlebotomist or blood collecting unit.

In embodiments where there is single patient temperature monitoring device associated with a number of blood processing units/patients, each blood collection unit 520, 620 may be connected to a group computing device 412 which can connect to the controller 522, 622 for each blood collecting unit 520, 620. Alternatively, the system may not include a group computing unit and the patient temperature monitoring device 102 may communicate directly with a controller associated with each blood collecting unit.

The group computing device 412, when used, may have a controller which is able to collect, store and analyze data from the patient temperature monitoring device 402 and each blood collection unit 520, 620. The group computing device 412 may be a part of a server, “the cloud” or a web-based network. In one embodiment, the group computing device 412 is at least one of the group consisting of a personal computer, tablet, laptop, or cellular phone. However, the group computing device 412 may be any such device capable of collecting data, processing data, analyzing data, and sending relevant information to the controllers 522, 622 and/or blood collection units 520, 620 associated with each patient.

Once new data from the patient temperature monitoring device 402 is generated, the information may be transmitted, downloaded or otherwise sent from the patient temperature monitoring device 402 to the group computing device 412 and then to one or more controllers 520, 620 associated with the blood collecting units 520, 620. The group computing device 412 may send a signal to any blood collecting unit 520, 620 or associated controller 522, 622 that an alarm should be generated, and the blood collection paused or stopped.

Before a typical blood collection procedure, a user or phlebotomist may program the controller 222 by the using the input 226 to add patient specific characteristics and parameters, such as a current or baseline temperature or temperatures for sections of the patients face or body. The patient is then phlebotomized using the vein access device. Whole blood is drawn into the system. During the blood draw, the patient temperature monitoring device 102 occasionally and/or at selected intervals will measure at least one temperature of the patient. The temperature may also be done continuously with the infrared camera 108. These measurements are compiled by the controller and analyzed in order to determine if a significant change in patient temperature has occurred, and a patient reaction is imminent or likely. The analysis may include comparing the measured temperatures to baseline temperature entered or measured before the blood draw. If the controller calculates a patient reaction is likely to occur, then the controller instructs the input and/or indicator to communicate with a phlebotomist or user that the procedure should be ended. The patient temperature monitoring device continues to measure the temperature and the controller 222 continues to analyze, calculate the probability of a reaction, and alert the phlebotomist, patient, or blood collecting unit 220 as needed until the blood draw is complete or stopped.

The measured temperatures include measured temperature for at least a section or sections of a patient's face and compare those measurements to previous measurements or different sections of the patient's face.

Thus, improved blood draw systems and methods have been provided. The system and method should provide for the ability to anticipate a patient reaction and prevent or minimize the reaction.

### Aspects

Aspect 1. A method for reducing patient reactions during a blood draw using a blood draw system with a blood collection unit and a controller, the method comprising: (a) establishing a baseline temperature for a patient; (b) initiating a blood draw; (c) measuring patient temperature with at least one patient temperature monitoring device during the blood draw for a measured patient temperature; (d) comparing the measured patient temperature to the baseline temperature; (e) predicting whether a patient reaction is likely to occur; (f) stopping the blood draw if a patient reaction is likely to occur; and (g) performing steps b-f until the blood draw is complete.

Aspect 2. The method of Aspect 1, wherein the temperature is a facial temperature.

Aspect 3. The method of Aspect 1, wherein establishing the baseline temperature includes measuring the temperature of the patient and setting that temperature as the baseline temperature.

Aspect 4. The method of Aspect 1, wherein establishing the baseline temperature includes inputting a baseline temperature into the blood draw system.

Aspect 5. The method of Aspect 4, wherein the baseline temperature is a range that includes minimum and maximum values.

Aspect 6. The method of Aspect 5, wherein predicting whether a patient reaction is likely to occur includes determining if the measured temperature is outside of the baseline temperature range.

Aspect 7. The method of Aspect 1, wherein predicting whether a patient reaction is likely to occur includes calculating a variation in temperature.

Aspect 8. The method of Aspect 7, wherein the variation in temperature is between the baseline temperature and the measured temperature.

Aspect 9. The method of Aspect 7, wherein the variation in temperature is between different sections of the patient's face.

Aspect 10. The method of any of Aspects 7-9, wherein predicting that a patient reaction will occur if the variation is above a set value.

Aspect 11. The method of Aspect 10, wherein the set value is 2° C.

Aspect 12. The method of any of the preceding Aspects, wherein at least one patient temperature monitoring device includes at least one camera.

Aspect 13. The method of Aspect 12, wherein at least one camera includes an infrared camera.

Aspect 14. The method of Aspect 12, wherein the at least one camera includes a digital camera.

Aspect 15. The method of Aspect 13 wherein the method further includes calibrating the infrared camera before initiating a blood draw.

Aspect 16. The method of any of the preceding Aspects, wherein the baseline and measured temperatures are taken at least one portion of the patient's face.

Aspect 17. The method of Aspect 16, wherein the at least one portion of the patient's face includes a forehead of the patient.

Aspect 18. The method of Aspect 1, wherein the patient temperature monitoring device includes an infrared camera and a digital camera and measuring the temperature of the patient during a blood draw includes mapping pixels from each camera on top of each other and capturing the temperature at those pixels.

Aspect 19. The method of Aspect 18, wherein the pixels are within a single section of the patient's face.

Aspect 20. The method of Aspect 18, wherein the pixels are within two sections of the patient's face.

Aspect 21. The method of Aspect 20, wherein the comparing the measured temperature to the baseline temperature includes comparing the two sections of the patient's face.

Aspect 22. A system for performing a blood draw procedure comprising: a vein access device; a fluid circuit comprising tubing connected to a vein access device and at least one blood collection container for collecting blood and/or blood components; at least one patient temperature monitoring device; an indicator; and

a controller configured to communicate with the patient temperature monitoring device and the indicator, wherein the controller is configured to: (i) establish a baseline temperature for a patient; (ii) instruct the patient temperature monitoring device to measure patient temperature during the blood draw and record the measured temperature; (iii) compare the measured temperature to the baseline temperature; (iv) predict whether a patient reaction is likely to occur; (v) initiate an alarm at the indicator in order to stop the blood draw if a patient reaction is likely to occur; and (vi) perform steps i-v. until the blood draw is complete.

Aspect 23. The system of Aspect 22, wherein the patient temperature monitoring device is a facial temperature monitoring device.

Aspect 24. The system of any of Aspects 22-23, wherein the patient temperature monitoring device includes at least one camera.

Aspect 25. The system of Aspect 24, wherein at least one camera includes an infrared camera.

Aspect 26. The system of Aspect 24, wherein the at least one camera includes a digital camera.

Aspect 27. The system of Aspect 22, wherein the patient temperature monitoring device includes an infrared camera and a digital camera.

Aspect 28. The system of Aspect 22, wherein the system further includes an additional patient parameter monitoring device.

Aspect 29. The system of claim 28, wherein the additional patient parameter monitoring device includes at least one of a blood pressure cuff, a pulse oximeter, and an electrocardiogram.

Aspect 30. A blood draw system comprising: (a) a plurality of blood collection units, each unit including: (i) a fluid circuit; (ii) at least one blood collection container; (a) a patient temperature monitoring device; (b) an indicator associated with each blood collection unit; and (c) a controller associated with each blood collection unit configured to communicate with the patient temperature monitoring device and an indicator, wherein the controller is configured to: (i) establish a baseline temperature for a patient; (ii) instruct the patient temperature monitoring device to measure patient temperature during the blood draw and record the measurements; (iii) compare the measured temperature to the baseline temperature; (iv) predict whether a patient reaction is likely to occur; (v) initiate an alarm at the indicator in order to stop the blood draw if a patient reaction is likely to occur; and (vi) perform steps i-v. until the blood draw is complete.

Aspect 31. The system of Aspect 30, wherein the patient temperature monitoring device is a facial temperature monitoring device.

Aspect 32. The system of any of Aspects 30-31, wherein the patient temperature monitoring device includes at least one camera.

Aspect 33. The system of Aspect 32, wherein at least one camera includes an infrared camera.

Aspect 34. The system of Aspect 32, wherein the at least one camera includes a digital camera.

Aspect 35. The system of Aspect 30, wherein the patient temperature monitoring device includes an infrared camera and a digital camera.

Aspect 36. The system of Aspect 30, wherein the system further includes an additional patient parameter monitoring device.

Aspect 37. The system of Aspect 30, further comprising a group computing unit configured to communicate with the controller associated with each blood collection unit.

Aspect 38. A method of calibrating an infrared camera including: (a) capturing an image of an object with a uniform temperature; (b) zooming in on the image to establish a real temperature; (c) zooming out on the image and measuring temperatures across the image; (d) formulating an adjustment equation to correct for temperature outside of the real temperature, based on location from a left side of the infrared camera; and (e) applying the adjustment equation to the camera.

Aspect 39. The method of Aspect 38, wherein the calibrating is performed before each camera use.

1. A method for reducing patient reactions during a blood draw using a blood draw system with a blood collection unit and a controller, the method comprising:
a. establishing a baseline temperature for a patient;
b. initiating a blood draw;
c. measuring patient temperature with at least one patient temperature monitoring device during the blood draw for a measured patient temperature;
d. comparing the measured patient temperature to the baseline temperature;
e. predicting whether a patient reaction is likely to occur;
f. stopping the blood draw if a patient reaction is likely to occur; and
g. performing steps c-f until the blood draw is complete.

2. The method of claim 1, wherein the temperature is a facial temperature.

3. The method of claim 1, wherein establishing the baseline temperature includes measuring the temperature of the patient and setting that temperature as the baseline temperature.

4. The method of claim 1, wherein establishing the baseline temperature includes inputting a baseline temperature into the blood draw system.

5. The method of claim 4, wherein the baseline temperature is a range that includes minimum and maximum values.

6. The method of claim 5, wherein predicting whether a patient reaction is likely to occur includes determining if the measured temperature is outside of the baseline temperature range.

7. The method of claim 1, wherein predicting whether a patient reaction is likely to occur includes calculating a variation in temperature.

8. The method of claim 7, wherein the variation in temperature is between the baseline temperature and the measured temperature.

9. The method of claim 7, wherein the variation in temperature is between different sections of the patient's face.

10. The method of claim 7, wherein predicting that a patient reaction will occur if the variation is above a set value.

11. The method of claim 10, wherein the set value is 2° C.

12. The method of claim 1, wherein at least one patient temperature monitoring device includes at least one camera.

13. The method of claim 12, wherein at least one camera includes an infrared camera.

14. The method of claim 12, wherein the at least one camera includes a digital camera.

15. (canceled)

16. The method of claim 1, wherein the baseline and measured temperatures are taken at at least one portion of the patient's face.

17. (canceled)

18. The method of claim 1, wherein the patient temperature monitoring device includes an infrared camera and a digital camera and measuring the temperature of the patient during a blood draw includes mapping pixels from each camera on top of each other and capturing the temperature at those pixels.

19. The method of claim 18, wherein the pixels are within a single section of the patient's face.

20. The method of claim 18, wherein the pixels are within two sections of the patient's face.

21. (canceled)

22. A system for performing a blood draw procedure comprising:
a vein access device;
a fluid circuit comprising tubing connected to a vein access device and at least one blood collection container for collecting blood and/or blood components;
at least one patient temperature monitoring device;
an indicator; and
a controller configured to communicate with the patient temperature monitoring device and the indicator, wherein the controller is configured to:
i. establish a baseline temperature for a patient;
ii. instruct the patient temperature monitoring device to measure patient temperature during the blood draw and record the measured temperature;
iii. compare the measured temperature to the baseline temperature;
iv. predict whether a patient reaction is likely to occur;
v. initiate an alarm at the indicator in order to stop the blood draw if a patient reaction is likely to occur; and
vi. perform steps i-v. until the blood draw is complete.

23. The system of claim 22, wherein the patient temperature monitoring device is a facial temperature monitoring device.

24. The system of claim 22, wherein the patient temperature monitoring device includes at least one camera.

25. The system of claim 24, wherein at least one camera includes an infrared camera.

26. The system of claim 24, wherein the at least one camera includes a digital camera.

27. (canceled)

28. (canceled)

29. (canceled)

30. A blood draw system comprising:
a. a plurality of blood collection units, each unit including:
i. a fluid circuit; and
ii. at least one blood collection container;
b. a patient temperature monitoring device;
c. an indicator associated with each blood collection unit; and
d. a controller associated with each blood collection unit configured to communicate with the patient temperature monitoring device and an indicator, wherein the controller is configured to:
vii. establish a baseline temperature for a patient;
viii. instruct the patient temperature monitoring device to measure patient temperature during the blood draw and record the measurements;
ix. compare the measured temperature to the baseline temperature;
x. predict whether a patient reaction is likely to occur;
xi. initiate an alarm at the indicator in order to stop the blood draw if a patient reaction is likely to occur; and
xii. perform steps i-v. until the blood draw is complete.

31.-39. (canceled)
